(19) United States
(12) Patent Application Publication
(10) Pub. No.: US 2013/0071873 A1
(43) Pub. Date: Mar. 21, 2013

(54) ARRAY MICROINJECTION APPARATUSES AND METHODS

(71) Applicant: Jan De Sonneville, Leiden (NL)

(72) Inventor: Jan De Sonneville, Leiden (NL)

(73) Assignee: UNIVERSITEIT LEIDEN, Leiden (NL)

(21) Appl. No.: 13/635,711

(22) PCT Filed: Mar. 18, 2011
(86) PCT No.: PCT/EP2011/001367

§ 371 Date: Dec. 3, 2012

(30) Foreign Application Priority Data
Mar. 19, 2010 (GB) 1004629.0

(51) Int. Cl. C12M 1/26; B29C 67/00

(52) U.S. Cl. CPC C12M 33/04; B29C 67/0011
USPC 435/29; 435/283.1; 435/375; 264/162

(57) ABSTRACT

An array microinjection apparatus comprises a substrate (10) defining an in-use upper surface (11) having formed therein a plurality of part-spherical recesses (12). Each of these includes an in-use upwardly facing opening (13), the dimensions of the recesses (12) being such that each said recess accommodates therein a single cell; or the dimensions of the recesses being such that each said recess accommodates therein a single embryo, the recesses (12) being arranged in the surface (11) in a regular pattern.

The invention relates to array microinjection apparatuses and methods.

Microinjection is an area of growing technological and commercial interest in which it is desired to cultivate, study, grow and/or modify biological material, such as but not limited to cells and embryos, using methods that involve injecting biological or chemical matter into very small living building blocks.

The bioscience community is particularly interested in being able to perform such activities at a single-cell or single-embryo level, since there are perceived advantages to studies carried out in the absence of influences from neighbouring cells, organs or embryos.

To this end numerous devices have been proposed that permit the cultivation of cells or embryos (referred to herein generally as “particles”) on an individual basis. The development of such devices has generally coincided with improvements since the 1950's in microscopy and in the creation and use of tools employed for the purpose of manipulating and/or modifying such minute examples of living matter.

In studies and experiments employing micron-scale particles it is important both from the standpoint of statistical robustness of the results and the capturing of data (which very often is essentially optical data) that large numbers of experiments are performed simultaneously under identical conditions. This has led to the development of devices in which very large numbers of particles of living matter can be treated, assayed or modified in a very small area that is only visible using high-magnification microscopes.

In some cases scaffolds are developed, from biologically inert materials or even materials such as collagen-based mixtures that positively promote the survival of particles, in which particles such as cells can be grown in a way that permits their expansion in three dimensions.

Although such devices are in many cases useful they are not suited to the study of all types of particle. On the contrary in some studies it is necessary to provide constraints on the growth of the particles in order to provide experimentally desirable conditions.

Patent application no GB 0904720.0, the content of which is incorporated herein by reference, discloses and claims an array of wells having sides that taper towards a bottom layer. A liquid displacement member is operable to displace liquid in the wells such that only a relatively small quantity of liquid surrounds the particles of interest during an experiment. One objective of using the well array of GB 0904720.0 is to ensure that concentrations of fluid surrounding living matter accurately mimic conditions in organisms.

Although the apparatus of GB 0904720.0 is extremely effective in generating reliable experimental results there remains a need for devices that provide improvements over existing technologies.

One area in which such improvements are actively desired is that of microinjection.

In this technique a very fine cannula is maneuvered so as to penetrate the phospholipid bilayer of a cell so as to permit injection of material (such as but not limited to DNA) into the nucleus of either a single cell or a cell that is part of the embryo of a eukaryote.

Another area in which microinjection is of interest concerns the injection of material into the yolk of an embryo.

When using a prior art cell cultivation apparatus however the researcher normally finds that the target cells or embryos are randomly located in the experimental apparatus. As a result the researcher must locate each target cell individually using a microscope, and perform injection operations typically on a cell-by-cell basis. This is time-consuming, and hence expensive. Also it can be frustrating for the researcher to complete, and this factor can reduce the accuracy of experiments significantly.

A further drawback of any apparatus requiring one-by-one identification and injection of cells, yolks or nuclei is that the injections typically are not performed consistently from one injection to the next. This can be for example because the angle of insertion of the injection cannula, the injection force and the depth of insertion can all vary significantly.

The invention seeks to address these and other problems and in addition provides a novel manufacturing technique for producing a microinjection apparatus.

According to the invention in a first aspect there is provided an array microinjection apparatus comprising a substrate defining an in-use upper surface having formed therein a plurality of part-spherical recesses each including an in-use upwardly facing opening, the dimensions of the recesses being such that each said recess accommodates therein a single cell; or the dimensions of the recesses being such that each said recess accommodates therein a single embryo, the recesses being arranged in the said surface in a regular pattern.

The employment of a substrate having a surface in which a regular pattern or grid-like array of single-cell or single-embryo recesses exists means that it is possible with accuracy to locate cells in the recesses.

This greatly improves the ability of the researcher accurately to inject cell nuclei without error; and also enormously speeds up the process of injecting large numbers of nuclei.

The latter advantage in turn derives from the fact that since the cell locations may be known (because the recess locations are determinable) it is possible to calculate the positions of large numbers of nuclei and inject them simultaneously.

Preferably the substrate is transparent or translucent.

The regular pattern of recesses furthermore opens the possibility of new forms of research such as studying the migration of cells. The grid-like array of recesses permits the starting and finishing locations in studies of migratory cells to be identified with accuracy. This in turn means that mobility studies can be made more quantitative than in the past.

A study method based on these principles is described and claimed herein.

Conveniently the regular pattern is a rectangular array. By “rectangular array” is meant any regular pattern of recesses in which the recesses identifiably are presented in rows and/or lines so as to permit the identification of their locations according to co-ordinates such as but not limited to Cartesian (x- and y-axis) co-ordinates that may be notionally superimposed on the array. An example is a honeycomb-type pattern of the recesses although other arrangements are possible within the scope of the invention.

Preferably the recesses are such as to permit part of a cell disposed therein, or part of an embryo disposed therein, to protrude beyond the opening.

This feature facilitates penetration of cells during microinjection, and also may provide advantages in the cultivation of cells and embryos (which as a result may be exposed to nutrients in a controlled manner).

Preferably the depth of each recess is chosen so as to permit washing of a particle therefrom.

This feature advantageously facilitates methods as defined hereinbelow. The precise depth may be chosen by the skilled worker, based on his knowledge of the typical sizes of the cells or embryos requiring study.

In order to assist in exposing particles to nutrients, etc., conveniently the apparatus includes a flowable medium, that is at least inert to biological material in the recesses, surrounding the recesses. In practice the flowable medium may be a cultivation medium containing e.g. nutrients and/or oxygen-carrying compounds that are required in order to preserve the existence of cells and/or promote their growth or division.

Practical embodiments of the apparatus of the invention may include a container surrounding the substrate so as to contain it. This in turn provides for containment of a flowable medium such as that described above.

Preferably the container includes at least one opening formed therein and one or more seals for releasably sealing the or each said opening. Such a feature may be strongly desirable in order to prevent contamination of experiments at times when injection is not required; and permit temporary access to particles supported in the recesses for the purpose of carrying out microinjection.

In order to facilitate such injection the apparatus of the invention optionally includes a plurality of injectors corresponding in number and location to the recesses on a one-to-one correspondence basis; and a carriage for the injectors that is capable of advancing the injectors towards and retracting the injectors from the recesses by means of the opening so as to permit selective injecting of material into the cells or embryos in the recesses.

Since the locations of the recesses of the array can all be known if the location of one of them is known the provision of a carriage that supports a plurality of injectors in the manner specified above provides for the simultaneous injection of large numbers of cells or embryos simultaneously.

An important feature of the invention is that the part-spherical shapes of the recesses tend to make the particles “self-centering” inside them. It follows that through accurately locating the centre of one of the recesses (as viewed from above, being the preferred direction from which the injectors may be moved towards the particles) it is possible to achieve good accuracy in injecting into the cell nuclei since these tend also to be located centrally in the part-spherical recesses.

A hemispherical shape to each recess is advantageous because it minimises the average distance between the centre of the particle and the centre of the recess.

Preferably each said injector is or includes a cannula that is capable of penetrating the nucleus of a cell and transfecting biological material thereinto.

Advantageously the substrate is made from, or includes, a gel including agarose, collagen or Matrigel™.

According to a second aspect of the invention there is provided a method of injecting biological matter including the steps of operating an apparatus as defined hereinabove so as to cause injection of biological or chemical material into one or more cell nuclei and/or embryo yolks in one or more said recesses in the substrate.

Preferably, in such a method, the step of operating the apparatus includes the step of advancing injectors towards the recesses so as to penetrate the nuclei of one or more cells and subsequently retracting the recesses therefrom, injection of biological material occurring following penetration of the one or more nuclei.

In particularly preferred embodiments of the invention the movement of the injectors occurs in a vertical direction while the substrate extends essentially horizontally. This has been found to improve the accuracy with which a researcher may inject cells, or may locate the yolk of an embryo.

Preferably the cells or embryos are adhered to the material of the substrate, the method further including the steps of:
treating the cells or embryos with an enzyme to cause their release from the substrate;
rinsing the released cells or embryos in order to dilute the enzyme;
centrifuging the released cells or embryos in order to permit their collection, and the removal of supernatant fluid;
adding flowable medium to collected cells or embryos in order to achieve a chosen medium concentration relative to the cells or embryos;
suspending the cells or embryos in the medium in recesses of the substrate; and
incubating the cells or embryos in the recesses.

It is also preferable that the enzyme is a protease, especially but not necessarily Trypsin.

In an alternative version of the method the cells or embryos are suspended in a flowable medium in the recesses of the substrate, the method including the further steps of:
centrifuging the suspended cells or embryos in order to permit their collection, and the removal of supernatant fluid;
adding flowable medium to collected cells or embryos in order to achieve a chosen medium concentration relative to the cells or embryos;
suspending the cells or embryos in the medium in recesses of the substrate; and
incubating the cells or embryos in the recesses.

One form of method for manufacturing an apparatus as defined herein includes the step of machining a substrate blank in order to form a plurality of recesses therein in a regular pattern.

According to another aspect of the invention there is provided a method of manufacturing an apparatus as defined hereinabove comprising the steps of forming or providing a forming member, including one or more protuberances that correspond in size and shape the dimensions of the recesses, of a relatively hard material; impressing the forming member into a mould blank of a relatively soft material a predetermined distance so as to define recesses therein; withdrawing the forming member from the resulting, formed mould blank; using the formed mould blank to manufacture a mould of a polymer material; and using the mould to form from a substrate blank the substrate of the apparatus including formed therein a plurality of recesses in a regular pattern.

In such a method of manufacture the recesses may, through judicious choice of the design of the mould, be arranged to be identical and arranged according to a preferred array pattern.

Preferably the method includes the further step of polishing the in-use upper surface of the formed mould blank. This has been found to be desirable in order to eliminate burrs or other projections that might result from the moulding process and interfere with the movement of particles into the recesses following seeding of the apparatus. As an alternative, repeated pressing of the forming member into the mould blank may eliminate this problem.

Conveniently the substrate blank is of or includes a settable material that hardens on setting, and the method includes initiating setting of the settable material. As examples one may consider collagen-based and/or agarose-based materials, for the substrate, that may be cured to a hardened state through the application of heat and/or by cooling as appropriate.

In order to ensure reliability of the formation of the recesses, preferably the protuberances are of equal length and size.

According to a fourth aspect of the invention there is provided a method of seeding an apparatus as defined hereinabove, the method comprising the steps of as necessary placing in a container a substrate defining an in-use upper surface having formed therein a plurality of part-spherical recesses each including an in-use upwardly facing opening, the dimensions of the recesses being such that each said recess accommodates therein a single cell; or the dimensions of the recesses being such that each said recess accommodates therein a single embryo, and the recesses being arranged in the said surface in a regular pattern; placing a flowable medium that is at least inert to living particles in the container so as to surround the recesses; and adding a plurality of particles to the interior of the container so as to move onto the substrate and thence individually into the insides of respective recesses. As desired the apparatus may be centrifuged or otherwise agitated in order to promote this process, although in some cases gravity alone is adequate.

As desired the flowable medium and the particles may be added simultaneously or separately.

To prevent formation of trapped air the substrate surface is preferably hydrophilic.

The provision of a regular pattern of the recesses means that the boundaries between adjacent recesses also are defined according to a regular arrangement. The boundaries may be designed, in accordance with the principles of the invention, so as to be relatively inhospitable to the particles such that they readily fall under gravity into the recesses. Once this has occurred the particles may be nurtured as desired with the positions of their nuclei being easily determined so they can readily be injected or studied en masse.

According to yet another aspect of the invention there is provided a method of observing cell migration including sending apparatus as defined herein with cells so that cells occupy recesses of the substrate thereof; applying a cell cultivation medium above the substrate; and visually observing migration of cells from one recess to or towards another, the locations of the recesses being indexable by reason of the regularity of their pattern.

In such a method the locations of the recesses may be identified using a Cartesian co-ordinate system. As a result it becomes straightforward to quantify the extent and direction of cell movement.

It is particularly desirable in the case of this cell migration observation method for the substrate material to be transparent or translucent.

By “inhospitable” is meant, for example, an environment in which the particles find it hard or impossible to bind to the material of the boundaries between the recesses. In consequence a relatively small energy input (such as that provided by gentle agitation of the substrate) is sufficient beneficially to cause the majority of the particles to fall into the recesses.

The listing or discussion of any apparently prior-published document in this specification should not necessarily be taken as an acknowledgement that the document is part of the state of the art or is common general knowledge.

There now follows a description of preferred embodiments of the invention, by way of non-limiting example, with reference being made to the accompanying drawings in which:

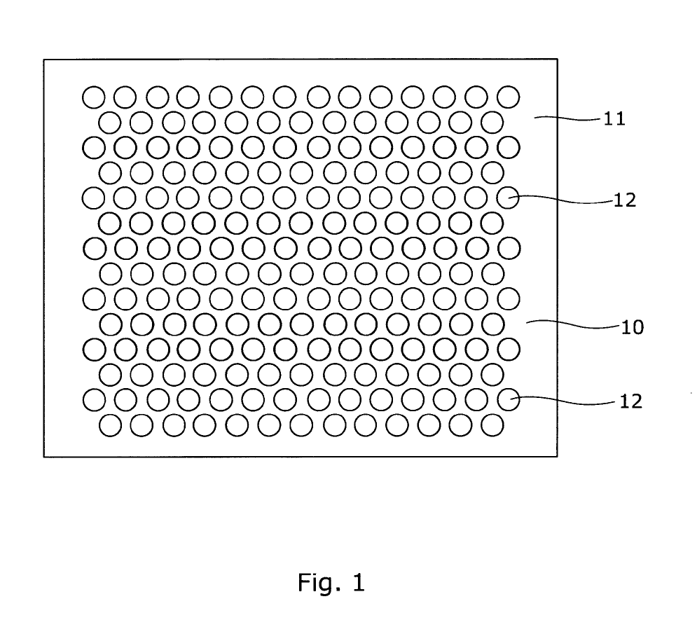

FIG. 1 is a plan view from above of a substrate, according to the invention, duplicates of which may be formed or placed in the bottom of a plurality of wells of a microwell plate;

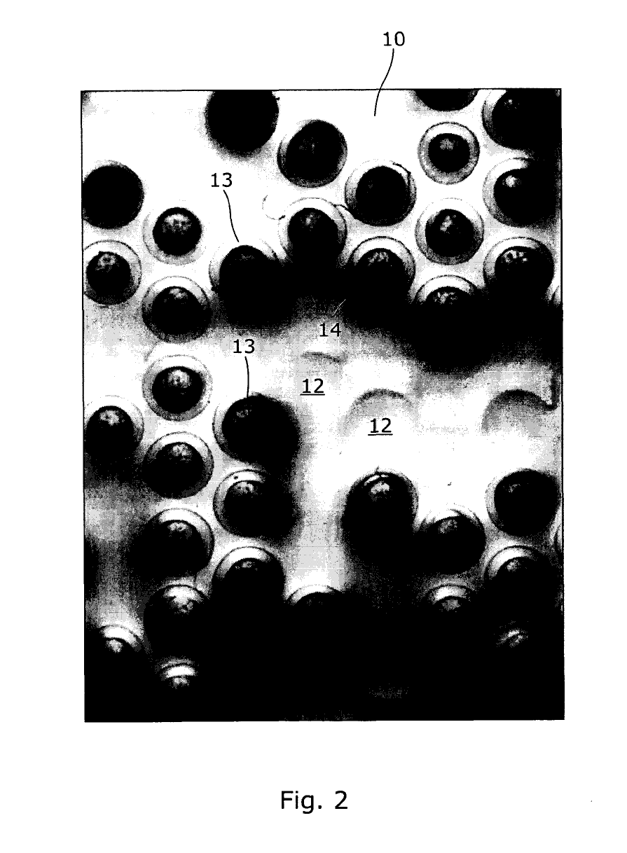

FIG. 2 is a photomicrograph taken from above at a higher magnification than FIG. 1 of the substrate showing individual recesses containing respective cells the nuclei of which are clearly visible;

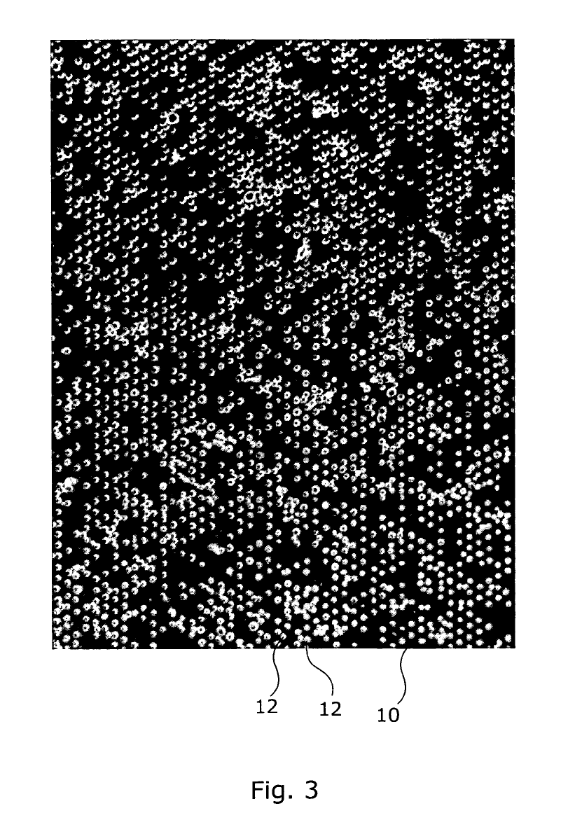

FIG. 3 is a plan view from above of the FIG. 2 substrate taken at a lower level of magnification in order to illustrate the extent of filling of the recesses by cells and/or embryos;

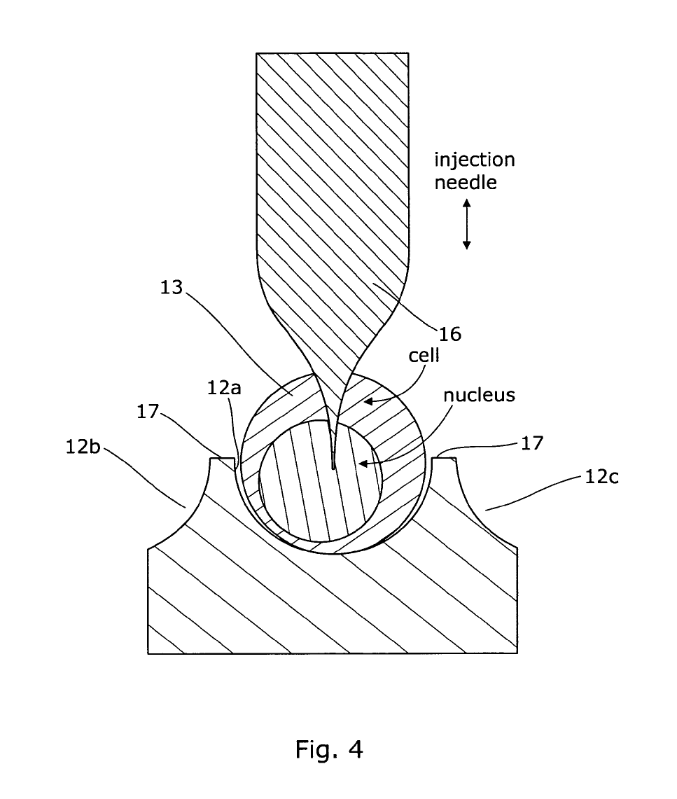

FIG. 4 is schematic, side-elevational, partly sectioned view showing microinjection of a cell in a recess and also illustrating one design of boundary between adjacent recesses; and

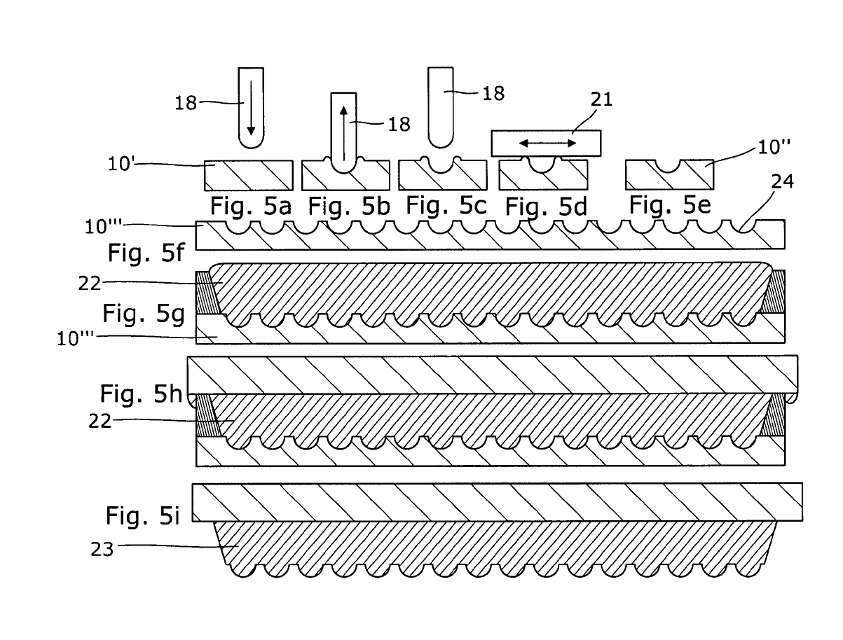

FIGS. 5a-5i schematically illustrate steps in a preferred process of manufacture of the substrate of FIGS. 1-3.

Referring to the figures an array microinjection apparatus includes a substrate 10 having formed in an in-use upwardly facing surface 11 in a regular, rectangular grid pattern defined by a plurality of recesses 12 hollowed into the upper surface from above.

In FIG. 1 the recesses 12 are dimensioned to suit the cultivation of zebrafish embryos and therefore each have a diameter of 1.3 mm. In the case of wishing to cultivate other particles other recess sizes may be chosen as desired.

In the embodiment shown the substrate 10 is rectangular although other shapes are possible within the scope of the invention. The substrate 10 is small enough to be placed lying horizontally (i.e. so that surface 11 faces upwards) in the bottom of a well in e.g. a titre plate such as a 96-well plate.

FIG. 2 shows a region of substrate 10 in a condition of use to cultivate cells 13 for study and/or experimentation. The recesses 12 in this figure contain individual HeLa cells. The diameter of each recess 12 in this case is approximately 15 micrometer.

In FIG. 2 the cell nuclei in themselves are not visible, but the cell structure creates a visibly darkened region 14 that is generally concentric with the region of each cell corresponding to the nucleus location.

The recesses 12 are part-spherical or part-spheroidal in shape. Their dimensions in the preferred embodiment shown are such that each of the recesses may accommodate a single cell. In another embodiment the recesses 12 may be sized so as to accommodate a single embryo of a eukaryote such as a zebra fish, carp or similar analogue of mammalian cell behaviour.

In more detail, each recess 12 terminates at its upper boundary in an opening the diameter of which is very slightly greater than the cell or embryo intended to be cultivated. As a result of this and of the part-spherical shape of the recesses the cells/embryos tend to self-centre in the recesses, such that their nuclei (which also are clearly visible in FIG. 2) are located approximately centrally. As explained in more detail below this permits highly accurate microinjection into the nuclei when required.

FIG. 3 shows a substrate similar to that of FIG. 2 having smaller recesses filled with HeLa cells. This illustrates the extent to which the cells 12 may occupy the array of recesses 10. As is apparent from FIG. 3 the density of cells in the recesses is high, with the result that a microinjection regime relying on advancement of cannulae towards the centres of the recesses is likely to cause injection of a large number of nuclei. Therefore in contrast with the prior art the apparatus of the invention is capable of efficiently permitting the injection of large numbers of nuclei in a short period.

Referring now to FIG. 4 a recess 12a is shown in side elevational, partly-sectioned view. In the embodiment illustrated the recess is hemispherical, although in other embodiments greater or smaller proportions of spheres or ovoids may be defined.

It is however preferred that at least a part of the contained cell 13 is exposed to the ambient environment surrounding the substrate 10. Assuming the ambient environment contains nutrients that are suitable to promote the existence of the cell 12 this arrangements ensures exposure of the cell phospholipid barrier to the nutrients which may then be absorbed by the cell.

The recess diameter is large enough to permit growth of the cell while being small enough to prevent more than one cell from occupying a single recess 12; and the ability to expose part of the cell permits three-dimensional growth.

FIG. 4 additionally shows an injection needle or cannula 16 injecting the nucleus of the cell 13. As is indicated by the arrows in FIG. 4 injection is achieved through a process of advancement and retraction of the cannula 16 in a vertical direction.

This has been found to be highly reliable because it is possible through this technique, once the location of at least one recess 12 in the array is known, accurately to cause entry of a plurality of needles supported in a rectangular grid pattern on a carriage into the nuclei of a large number of cells in the recesses 12.

This is in turn achieved through the use of an array of cannulae that are supported in a grid pattern that corresponds in number and location to the pattern of the recesses.

In a practical set-up the substrate 10 would first be inserted in the horizontal orientations shown into a well that would serve as a container for the substrate. The well preferably would be sealable in a manner that is openable to permit injection in the manner outlined above.

A flowable medium, such as a cell nutrient mixture, would then be filled into the well to chosen depth covering the substrate.

The medium would be pre-seeded or subsequently seeded with cells 13 or, if desired, embryos. These would fall under gravity onto the substrate 10. Some would immediately fall into recesses such as recess 12a whereas others would lodge on the lands 17 separating adjacent recesses 12a, 12b, 12c etc from one another.

The lands 17 are designed to be relatively thin so that any cell lodged on one of them essentially is in unstable equilibrium. Such cells therefore may easily be caused to fall into adjacent recesses e.g. as a result of gentle agitation of the substrate 10.

The cells or embryos are then available suspended in the medium in the recesses for study and experimentation.

As an alternative to seeding the liquid in a well with cells/embryos the substrate may be pre-seeded, before insertion into the well, through a process of adhering the cells to the substrate in the recesses.

When the cells/embryos are in suspension the observation/experimentation technique may include numerous further steps preferred examples of which include:
centrifuging the suspended cells or embryos in order to permit their collection, and the removal of supernatant fluid;
adding flowable medium to collected cells or embryos in order to achieve a chosen medium concentration relative to the cells or embryos;
suspending the cells or embryos in the medium in recesses of the substrate; and
incubating the cells or embryos in the recesses.

Preferably the centrifuging takes place at a low enough speed as not to influence the cells or embryos in any adverse manner.

When the cells/embryos are adhered to the material of substrate 10 it is in addition desirable to release the cells and adjust the nutrient medium concentration before initiating incubation of the injected cells/embryos. This may be achieved by treating the cells/embryos in the recesses with e.g. Trypsin; and then washing the trypsinized particles in order to reduce the Trypsin concentration before performing the centrifuging step mentioned above.

Biological and/or chemical material that is suitable for injection into cells or embryos using the technique of the invention includes but is not limited to:
cells such as cancer cells, from the same type of organism as the investigated cells/embryos or from other organisms/cell lines;
bacteria causing disease (e.g. TB bacteria);
proteins for example that interact with membrane receptors or that visualise other protein (e.g. fluorescently labelled antibody proteins);
medicine compounds;
liposomes
beats or particles releasing protein or medicine compounds;
marker beats, such as antibody coated nanoparticles or other non-biological material.

In yet a further version of the method of the invention the apparatus is used to inject into the chorion of embryos.

Broadly, in accordance with the invention, there are two options for constructing the substrate 10:
1) Direct machining of the substrate in which half-spherical holes are milled out of a suitable material. Such suitable material is preferably hydrophilic. The hydrophilicity can if desired be altered by coating of a hydrophobic material. The substrate is preferably transparent as to permit viewing of the injection process from underneath using a microscope. (As the injection is preferably done in a vertical direction, there is no space on top for a microscope.)
   Preferably the refractive index of the substrate material is close to the refractive index of the flowable medium, such that the image is not significantly altered, when viewing through the half-spherical recess.
2) Machining of a positive mould by using CNC milling of e.g. a metal, then creating a negative copy, which is subsequently used to mould again a positive substrate. The advantage of this method is that gel on a flat glass substrate can be used as the final substrate material. As gel is 98/99 percent flowable medium, the refractive index is almost the same as the medium, and this then facilitates a perfect view from underneath the substrate of the injection (and calibration) process.

Referring now to FIGS. 5a to 5i a process of making the substrate 10 schematically is illustrated.

In this regard it is notoriously difficult to produce e.g. 10 micron diameter recesses using prior art techniques. Techniques of photolithography, for example, can in practice only produce recesses having significant spacings between them, despite the fact that in theory photolithography can produce small holes. This is because the photoresist used in such techniques requires a significant amount of material to attach to. When the holes are very small, however, the amount of substrate material between adjacent recesses becomes insufficient to assure fixing in place of the photoresist.

In FIG. 5a a mould blank 10′ is prepared as e.g. a small rectangular sheet of soft metal such as but not limited to brass or aluminium.

Such materials are relatively soft and hence easily deformed.

A forming member in the form of a relatively high-hardness rod 18 made e.g. from hardened steel is advanced from above as indicated by the vertical arrow in FIG. 5a into the horizontally extending sheet 10′ so as to imprint a hemispherical or other shape recess 12′ thereinto. The rod 18 is then withdrawn as shown by the arrow in FIG. 5b to leave the recess 12′ formed in the upper surface of the substrate 10′ (FIG. 5c).

This process may leave burrs 19 or other imperfections. Repetition of the steps of FIGS. 5a and 5b can remove such defects. As an alternative optional step as shown in FIG. 5d the burrs may be removed using a polishing tool 21. The resulting imprinted metal sheet (mould negative) 10″ is shown in FIG. 5e.

The steps of FIGS. 5a to 5e are repeated multiple times in order to create a mould negative 10′″ as shown in FIG. 5f having a regular pattern of recesses 12′ formed therein.

In an alternative method a large number of the rods 18 may be secured on a jig so as to be advanced simultaneously into the substrate blank 10′. Assuming the rods 18 were arranged in the pattern of recesses desired in the substrate this would result in the instantaneous formation of the entire recess pattern.

Once the negative 10″ is formed a polymer material such as polydimethylsiloxane (PDMS) or polyurethane is poured in a layer 22 into it (FIG. 5g), and pressed and cured (FIG. 5h) in order to create a positive mould member 23 having protrusions 24 in the pattern of recesses required in the substrate 10 of the apparatus. The mould member 23 may be used as often as desired (until it wears out) in order to imprint substrate blanks made of e.g. collagen, Matrigel™ or agarose and make the substrates 10 described above with high accuracy despite the small recess diameters required.

Although the preferred manufacturing method described above employs cylindrical rods 18 having rounded ends that produce the hemispherical recesses visible for example in FIG. 4, other rod shapes (that need not be circular in cross-section and need not have rounded ends) may as desired be employed.

In an alternative approach the material of the substrate 10 may be micromachined (e.g. milled) directly in order to create the desired pattern of recesses.

One further form of use of the apparatus of the invention is as follows:

Cells are oriented in the 2D honeycomb grid (i.e. the recess pattern) immersed in an agarose gel. On top of the cells another layer of matrigel or collagen gel is added to allow cells to migrate through this top gel. As agarose cannot be dissolved by a cell's protein the bottom layer is not suitable for migration. Agarose is nevertheless needed to give enough stiffness in order to stabilise the grid structure.

The grid pattern gives the opportunity to study cell migration and/or adhesion processes in a quantitative manner.

Agarose alone is not suitable for cells to adhere and grow. Therefore other components such as collagen or other cell matrix material should be mixed or added in.

Overall the apparatuses and methods described herein are capable of greatly improving the efficiency and speed of microinjection operations and the precision of manufacture of microinjecting array substrates.

1. An array microinjection apparatus comprising a substrate defining an in-use upper surface having formed therein a plurality of part-spherical recesses each including an in-use upwardly facing opening, the dimensions of the recesses being such that each said recess accommodates therein a single cell; or the dimensions of the recesses being such that each said recess accommodates therein a single embryo, the recesses being arranged in the said surface in a regular pattern, characterised in that the substrate is made from, or includes, a gel including one or more of agarose, collagen or Matrigel™.

2. An apparatus according to claim 1, wherein the regular pattern is a rectangular array.

3. An apparatus according to claim 1, wherein the recesses are such as to permit part of a cell disposed therein, or part of an embryo disposed therein, to protrude beyond the opening.

4. An apparatus according to claim 1, wherein the depth of each recess is chosen so as to permit washing of a particle therefrom.

5. An apparatus according to claim 1, including a flowable medium, that is inert to biological material in the recesses, surrounding the recesses.

6. An apparatus according to claim 1, including a container surrounding the substrate so as to contain it.

7. An apparatus according to claim 6, wherein the container includes at least one opening formed therein and one or more seals for releasably sealing the or each said opening.

8. An apparatus according to claim 1, including a plurality of injectors corresponding in number and location to the recesses on a one-to-one correspondence basis; and a carriage for the injectors that is capable of advancing the injectors towards and retracting the injectors from the recesses by means of the opening so as to permit selective injecting of material into the cells or embryos in the recesses.

9. An apparatus according to claim 8, wherein each said injector is or includes a cannula that is capable of penetrating the nucleus of a cell and transfecting biological material thereinto.

10. A method of injecting biological matter including the steps of operating an apparatus according to claim 8, so as to cause injection of biological material or chemical into one or more cell nuclei and/or embryo yolks in one or more said recesses in the substrate.

11. A method according to claim 10, wherein the step of operating the apparatus includes the step of advancing injectors towards the recesses so as to penetrate the nuclei of one or more cells and subsequently retracting the recesses therefrom, injection of biological or chemical material occurring following penetration of the one or more nuclei.

12. A method according to claim 11, wherein the cells or embryos are adhered to the material of the substrate, the method further including the steps of:
treating the cells or embryos with an enzyme to cause their release from the substrate;
rinsing the released cells or embryos in order to dilute the enzyme;
centrifuging the released cells or embryos in order to permit their collection, and the removal of supernatant fluid;
adding flowable medium to collected cells or embryos in order to achieve a chosen medium concentration relative to the cells or embryos;
suspending the cells or embryos in the medium in recesses of the substrate; and
incubating the cells or embryos in the recesses.

13. A method according to claim 12 wherein the enzyme is a protease, especially Trypsin.

14. A method according to claim 11, wherein the cells or embryos are suspended in a flowable medium in the recesses of the substrate, the method including the further steps of:
centrifuging the suspended cells or embryos in order to permit their collection, and the removal of supernatant fluid;
adding flowable medium to collected cells or embryos in order to achieve a chosen medium concentration relative to the cells or embryos;
suspending the cells or embryos in the medium in recesses of the substrate; and
incubating the cells or embryos in the recesses.

15. A method of manufacturing an apparatus according to claim 1, comprising the step of machining a substrate blank in order to form a plurality of recesses therein in a regular pattern.

16. A method of manufacturing an apparatus according to claim 1, comprising the steps of forming or providing a forming member, including one or more protuberances that correspond in size and shape the dimensions of the recesses, of a relatively hard material; impressing the forming member into a mould blank of a relatively soft material a predetermined distance so as to define recesses therein; withdrawing the forming member from the resulting, formed mould blank; using the formed mould blank to manufacture a mould of a polymer material; and using the mould to form from a substrate blank the substrate of the apparatus including formed therein a plurality of recesses in a regular pattern.

17. A method according to claim 16, including the further step of polishing the in-use upper surface of the formed mould blank.

18. A method according to claim 16, wherein the protuberances are of equal length and size.

19. A method of seeding an apparatus according to claim 1, comprising the steps of as necessary placing in a container a substrate defining an in-use upper surface having formed therein a plurality of part-spherical recesses each including an in-use upwardly facing opening, the dimensions of the recesses being such that each said recess accommodates therein a single cell; or the dimensions of the recesses being such that each said recess accommodates therein a single embryo, and the recesses being arranged in the said surface in a regular pattern; placing a flowable medium that is at least inert to living particles in the container so as to surround the recesses; and adding a plurality of particles to the interior of the container so as to move onto the substrate and thence individually into the insides of respective recesses.

20. A method of observing cell migration including seeding an apparatus as defined in claim 1, with cells so that cells occupy recesses of the substrate thereof; applying a cell cultivation medium above the substrate; and visually observing migration of cells from one recess to or towards another, the locations of the recesses being indexable by reason of the regularity of their pattern.
